(19) United States
(12) Patent Application Publication
(10) Pub. No.: US 2008/0249475 A1
(43) Pub. Date: Oct. 9, 2008

(54) TROCAR SEAL

(75) Inventors: Jeremy J. Albrecht, Ladera Ranch, CA (US); Donald L. Gadberry, San Clemente, CA (US); Gary M. Johnson, Mission Viejo, CA (US)

Correspondence Address: APPLIED MEDICAL RESOURCES CORPORATION, 22872 Avenida Empresa, Rancho Santa Margarita, CA, 92688, US

(73) Assignee: APPLIED MEDICAL RESOURCES CORPORATION, Rancho Santa Margarita, CA (US)

(21) Appl. No.: 11/677,994

(22) Filed: Feb. 22, 2007

Related U.S. Application Data
(60) Provisional application No. 60/775,840, filed on Feb. 22, 2006.

(51) Int. Cl. A61M 5/178

(52) U.S. Cl. 604/167.06; 604/167.01

(57) ABSTRACT

A trocar seal removably connected to a trocar cannula is provided that comprises an instrument seal and a zero seal with the zero seal connected to the instrument seal. A cover is connected to the instrument seal and a sleeve extends from the cover distally towards the end of the instrument seal.

### CROSS-REFERENCE TO RELATED APPLICATIONS

This application claims the benefit of U.S. Provisional Application No. 60/775,840 filed Feb. 22, 2006, the disclosure of which is hereby incorporated by reference as if set forth in full herein.

### BACKGROUND

The present invention generally relates to surgical access devices and, more specifically, to removable trocar seals.

Laparoscopic surgery is commonly performed using trocars having seals which provide instrument access across an abdominal wall and into a gas pressurized abdominal cavity. The functional requirements of such seals can be many and varied. Additionally, as laparoscopic surgery is an evolving modality, significant changes in instrumentation have challenged even the best trocar seals presently in use. However, in some applications the reduction of components is desired. The minimization of components can reduce manufacturing and assembly costs. Also, as some or parts of the components can be disposable, the amount of replaceable components can be reduced.

### SUMMARY

Generally, the present invention provides surgical access port or trocar with a low-profile seal assembly releasable from a cannula.

In one aspect, a trocar seal comprises an instrument seal, a zero seal and a cover. The instrument seal has a proximal end and a distal end. The zero seal has a proximal end and a distal end. The proximal end of the zero seal is connected to the proximal end of the instrument seal. The cover is connected to the proximal end of the instrument seal and a sleeve is monolithically formed with the cover and extends from the cover towards the distal end of the instrument seal.

In one aspect, a trocar seal comprises an instrument seal, a zero seal and a cover. The instrument seal has a proximal end and a distal end. The zero seal has a proximal end and a distal end. The proximal end of the zero seal is connected to the proximal end of the instrument seal. A cover is connected to the proximal end of the instrument seal. The cover has a substantially cylindrical sleeve extending from the cover and into the instrument seal and the cover has snaps extending from the cover parallel to the longitudinal axis and has projections extending orthogonal from the snaps towards the instrument seal.

In one aspect, a trocar seal comprises an instrument seal, a zero seal and a cover. The zero seal is nested in the instrument seal. The cover is connected to the instrument seal having means for protecting the instrument seal. The cover is arranged to be removably connected to a cannula by a means for restricting axial movement of the cover and for allowing rotational movement of the cover relative to the cannula.

Many of the attendant features of the present invention will be more readily appreciated as the same becomes better understood by reference to the foregoing and following description and considered in connection with the accompanying drawings in which like reference symbols designate like parts throughout.

### BRIEF DESCRIPTION OF THE DRAWINGS

FIG. 1 illustrates an exploded view of one aspect of a trocar in accordance with various aspects of the present invention;

FIG. 2 illustrates a cross-sectional view of one aspect of a seal assembly in accordance with various aspects of the present invention;

FIGS. 3-4 illustrate perspective views of aspects of a seal assembly in accordance with various aspects of the present invention;

FIG. 5 illustrates a cross-sectional view of one aspect of a seal assembly and cannula in accordance with various aspects of the present invention;

FIGS. 6-7 illustrate perspective views of aspects of a seal cover and sleeve in accordance with various aspects of the present invention;

FIG. 8 illustrates a cross-sectional view of one aspect of a seal assembly in accordance with various aspects of the present invention;

FIG. 9 illustrates a cross-sectional view of one aspect of a seal cover and sleeve in accordance with various aspects of the present invention; and

FIG. 10 illustrates a cross-sectional view of one aspect of a seal assembly and cannula in accordance with various aspects of the present invention.

### DETAILED DESCRIPTION

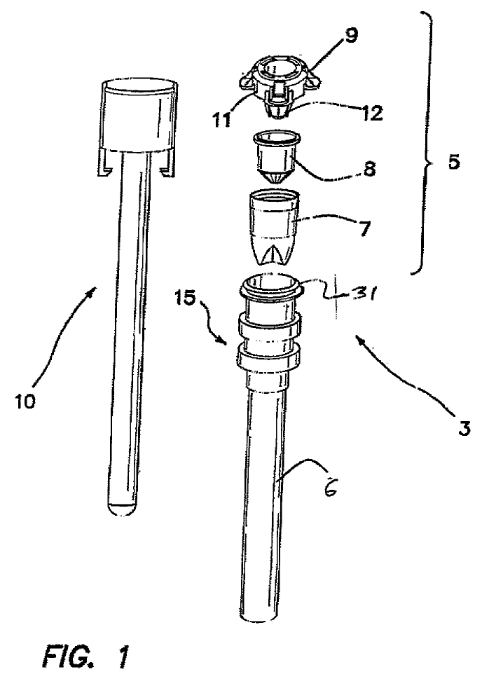

In FIG. 1, a surgical access port 3, e.g., a trocar, is provided. The trocar, for example, is used during minimally invasive surgery to provide an access channel into the body through which a surgeon may insert other medical instruments and/or an obturator 10. In particular, the trocar is configured to access a body cavity and to maintain positive pressure at its distal end to prevent loss of surgical insufflation gas such as carbon dioxide used, for example, in laparoscopic procedures to insufflate the body cavity.

In one aspect, the access port 3 comprises a trocar seal or seal assembly 5 and a cannula 6. The seal assembly is configured to sealingly engage surgical instruments of various diameters, which ultimately is inserted through the cannula, to prevent loss of surgical gas during use of such instruments. The seal assembly in one aspect also comprises a zero seal such that the loss of surgical gas is prevented when surgical instruments are not inserted or removed from the seal assembly and/or cannula. In one aspect, the seal assembly is releasably attachable to the trocar cannula. As such, this may allow the seal assembly to be attached to various types of similarly configured cannula of different lengths, shapes, colors and/or having other features, e.g., disposable, reusable and/or flexible. This can also allow the seal to be removed during surgery to enable the extraction of tissue specimens through the trocar without contact with the seal assembly.

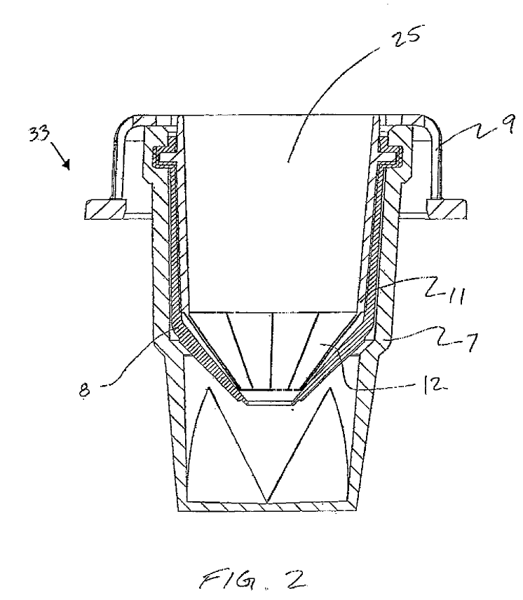

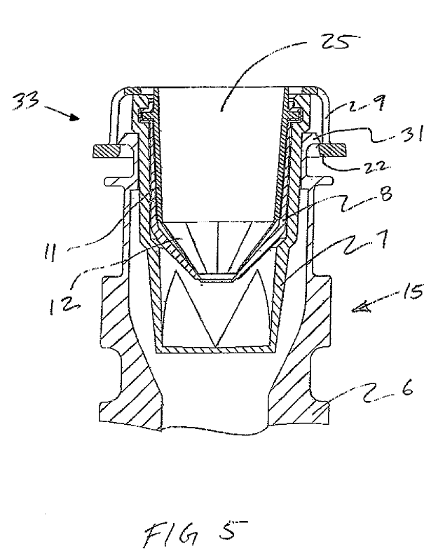

Referring now also to FIGS. 2-5, the seal assembly in one aspect comprises an instrument or septum seal 8 and a zero seal 7. The instrument seal forms a seal to maintain pneumoperitoneum around instruments inserted through the seal while the zero seal forms a seal to maintain pneumoperitoneum when no instrumentation is present in the seal assembly and/or cannula. In one aspect, the septum seal and/or zero seal is fixed in place. For example, the septum seal and/or zero seal do not swing or pendulate to a large degree, if any, relative to the cannula, seal sleeve 11 or seal cover 9. The zero seal 7 in combination with the septum seal 8 enables the seal assembly 5 and cannula 6 to accommodate a wide range of instrumentation diameters while minimizing the overall size of the seal assembly and cannula.

By fixing or minimizing the septum seal and/or zero seal swing or movement, the amount of space used to accommodate the seal assembly within the seal housing and/or the cannula to enable such movement of the septum seal and/or the zero seal is avoided or reduced. The additional accommodating space can result in a seal assembly with a relatively large axial length and overall diameter. As such, the fixed septum seal and/or fixed zero seal allows a seal housing and/or cannula to be sized with a reduced axial length and/or a reduced diameter relative to other seal assemblies. In one aspect, one end of the cannula has an enlarged end or section 15 to accommodate or house portions the seal assembly, e.g., largely portions of seal sleeve 11, septum seal 8 and/or zero seal 7. In one aspect, a separate seal housing is provided to accommodate the seal assembly and to assist in attaching the seal assembly to the cannula.

The zero seal 7 in one aspect also acts as a seal between the cannula 6 and the seal sleeve 11. The seal is generated by the compression of a proximal flange of the zero seal 7 between the top of the cannula and the underside of the seal sleeve. The septum seal 8 is completely contained or encompassed by the zero seal. In one aspect, the zero seal is a single or double duckbill valve. The seal assembly can accommodate a range of instruments having a diameter of 8 to 12 mm. However, instruments of less than 8 mm in diameter, such as 5 mm, can be accommodated when the instrument is centered in the seal assembly and cannula without torque being applied onto the instrument relative to the axis of the cannula. For example, a 5 mm diameter instrument can be used with the seal assembly in conjunction with a daVinici robot, which holds the instrument in the center of the cannula and prevents torque from being applied to the instrument. Different instrument sizes may also be accommodated from beyond 12 mm or below 8 mm by adjusting the dimensions of the septum seal and/or zero seal. For example, by increasing the diameter of the septum seal, the drag force produced during axial movement of inserted instrumentation through the seal is reduced to accommodate larger instruments. However, although large instruments may create a seal with the septum seal when in use, smaller diameter instruments may not be able to create a seal with the septum seal.

A reduction in instrumentation drag force for trocar seals can assist with positioning of instrumentation relative to the operative tissue A reduction in instrumentation drag force also decreases the likelihood that the seal assembly and/or cannula will be dislodged from a body wall during withdrawal of an instrument. A reduction in instrumentation drag force can also result in less fatigue for the surgeon, for example, during complex surgical procedures. As such, in one aspect, the dimensions of the seal assembly can be optimized to reduce instrumentation drag force for a range of instruments. For example, the inner diameter of the septum seal closely corresponds to the diameter of the surgical instrument.

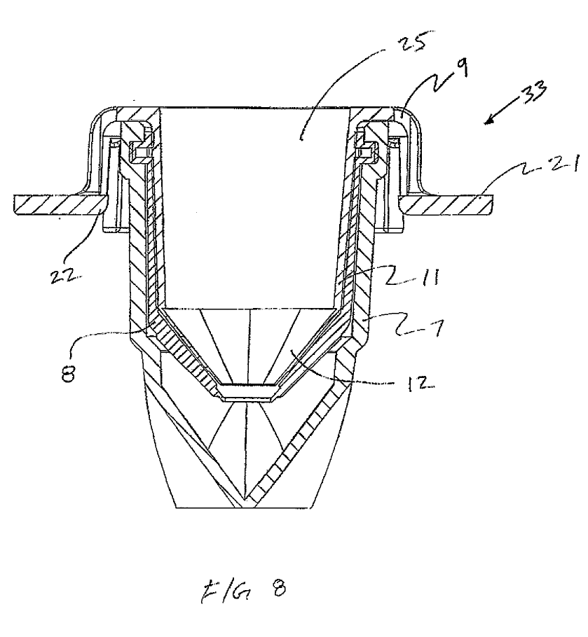

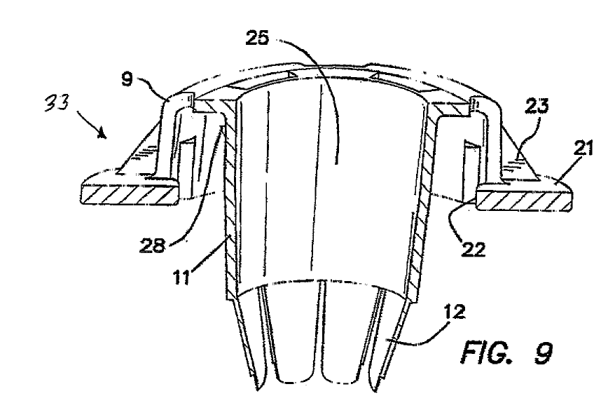

As shown in FIGS. 2, 5, 8-9, in one aspect, a seal sleeve 11 is juxtaposed to the septum seal 8. The sleeve in one aspect is substantially fixed and/or generally rigid. In one aspect, the sleeve is a long tubular or cylindrical channel, which extends from the aperture 25 in the seal cover 9 to proximally above the aperture in the septum seal. The sleeve can serve to align or guide instrumentation during insertion and manipulation, e.g., restricting the degree to which instrumentation can be manipulated. The sleeve also decreases the likelihood that instrumentation can catch, tear, or otherwise disrupt the septum seal and/or duckbill valve. Also, the sleeve being fixed can restrict the degree to which an inserted instrument can be manipulated. As such, the degree of pendulous movement utilized to accommodate instrumentation with a wide range of diameters is decreased. However, the size of the seal assembly can be more compact Examples of seal sleeves and shielded septum seals are described in U.S. patent application Ser. No. 11/000,123, filed Nov. 30, 2004, and U.S. Provisional Patent Application No. 60/529,455, filed Dec. 12, 2003, the entire disclosures of which are hereby incorporated by reference as if set in full herein.

In one aspect, the seal sleeve 11 is integrated with or otherwise coupled to the seal cover 9 and defines an instrumentation channel from the aperture of the seal cover 9 through the seal sleeve 11. The seal sleeve in one aspect is separate from the seal cover 9 and attached to the seal cover through lateral projections snapping into mating slots on disposed on the seal cover 9. The seal sleeve 11 and the seal cover 9 in one aspect are integrated forming a monolithic structure. The seal sleeve, in one aspect, acts as a shield to protect the elastomeric septum seal and/or zero seal of the seal assembly 5. In one aspect, the seal sleeve comprises of low-density polyethylene (LDPE), high-density polyethylene (HDPE), nylon, polypropylene or any combination thereof. The seal sleeve 11 is generally cylindrical extending from the seal cover 9. Extending from the seal sleeve away from the seal cover are one or more leaflets 12 through which an aperture is disposed or defined. In one aspect, the leaflets extend radially from the seal sleeve 11 towards the center or longitudinal axis of the sleeve or seal assembly. By reducing the number of leaflets provided, the potential for an instrument to bind between or bypass through the leaflets may increase. By increasing the number of leaflets 12, the width of individual leaflets may decrease which can reduce the force utilized to deflect the leaflet 12 when an instrument is inserted/removed from the seal, e.g., potentially reducing binding of the instrument in the seal assembly.

As the length of the leaflet 12 is increased the force required to deflect the leaflet during instrument removal is decreased. However, lengthening the leaflet may increase the axial height of the seal, which may reduce the “working length” of the surgical instruments being used. As the cross-sectional area is increased, the leaflet 12 may become rigid or stiffer and may be harder to deflect, but may also increase the durability of the leaflet and the protection that the leaflets can provide to the septum seal and/or zero seal.

The seal assembly 5 in one aspect can be detached or removed from the cannula 6 and attached or re-attached to the cannula for example before, during or after a surgical procedure. During surgery, for example, small tissue specimens may be extracted from a body cavity through the trocar cannula to enable pathological analysis of the tissue specimen. Avoiding or minimizing withdrawal of delicate tissue specimens through a seal assembly enhances maintenance of the integrity of the tissue specimen. As such, the seal assembly 5, which is removable from a cannula to enable extraction of tissue specimens from a body cavity while maintaining the integrity of the tissue specimen is provided. The seal assembly 5 also re-attaches to the cannula 6 after its initial removal during a surgical procedure. The seal assembly being removable from a cannula enables rapid de-insufflation of an insufflated body cavity. For example, towards the end of a laparoscopic surgical procedure, one of the steps involves the release of the insufflation gas such as carbon dioxide from the peritoneal cavity of the patient. This can be done by opening one or more stopcock valves provided with the seal assembly and/or cannula. The flow rate through the stopcock valves, however, can be slow with regard to evacuation of the carbon dioxide from the peritoneal cavity and therefore the time expended to evacuate the insufflation gas can be excessive or more than desired.

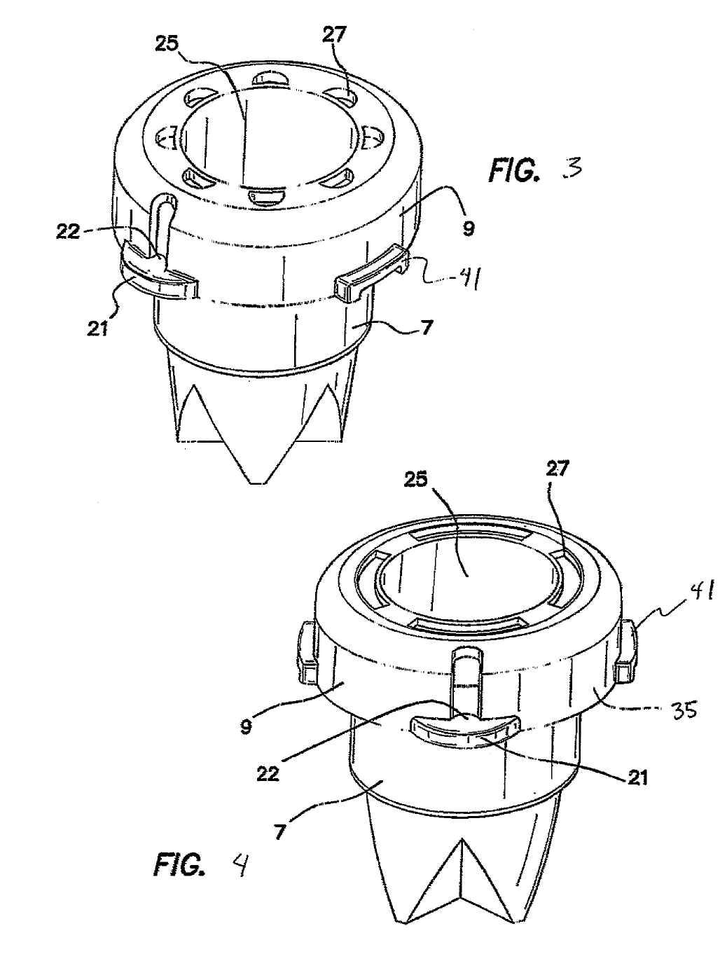

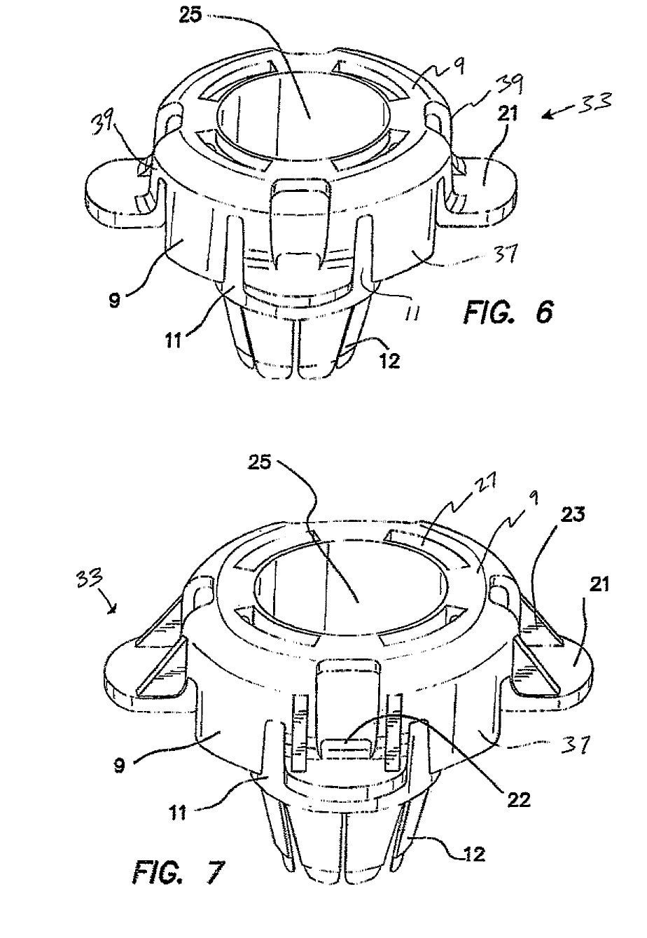

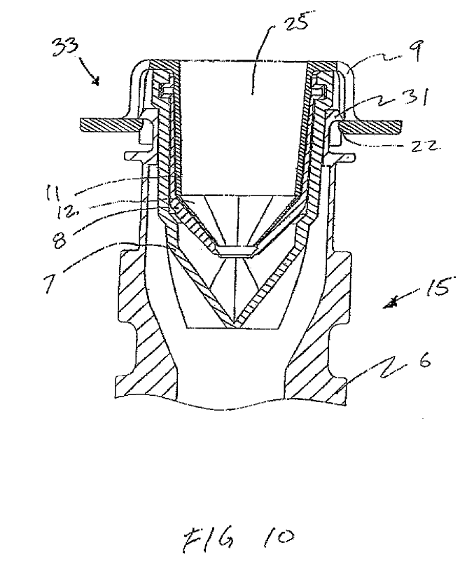

As shown in FIGS. 3-7 and 9-10, in one aspect, the attachment of the seal assembly 5 to the cannula 6 is provided by one or more snaps 33. In one aspect, four snaps engage the outside periphery of the cannula 6. The snap has a projection or hook 22 that engages an outer lip, shelf, slot or flange 31 extending along a portion or completely around the cannula 6. By applying a downward force or pressure on the cover 9, the hook releasably connects and seals to the outer periphery of the cannula. By applying an outward force or pressure on the snaps, the seal assembly is detached from the cannula. In one aspect, the retention force, the force required to remove the seal assembly 5 from the cannula 6, can be increased or decreased as desired. For example, gussets 23 extending along the cover 9 provide a retention force on an average of 12 lbs to 38 lbs. Also, for example, extending the sidewall 35 along the seal cover 9 to maintain a continuous outer periphery with the snaps can increase retention force. One or more sidewalls 37 along the seal cover 9 disposed on the outer periphery between the snaps or slots within the continuous sidewall or surface of the seal cover can also decrease the retention force of the seal cover 9.

The hooks or projections on the snaps 33 maintain the axial position of the seal assembly 5 on the cannula 6 and prevent axial dislodgment of the seal assembly 5 from the cannula 6. The snaps in one aspect have or are attached to arms 39. The arms or snaps are tapered and act as leaf springs during attachment of the seal assembly 5 onto the cannula 6. During attachment of the seal assembly 5 onto the cannula 6, the arms 39 flex outward until the distal ends of the arms reach slots or ledge 31 of the cannula 6. Once the distal ends of the arms 39 reach the cannula flange, the arms spring inward such that the hooks or projections 22 overhang the mating ledges 31 on the cannula 6. When an axial force is applied to the seal assembly relative to the cannula, the hooks 22 further engage the flange 31 on the cannula 6 and resist removal of the seal assembly 5 from the trocar cannula 6.

To remove the seal assembly 5 from the cannula 6, in one aspect, finger tabs 21 extending from portions of the periphery of the seal assembly are lifted or pivoted away from the cannula 5 causing the snaps 33 to move outward resulting in the disengagement of the hooks from the cannula flange 31. The seal assembly 5 can be then removed from the cannula 6 by applying an axial force to the seal assembly relative to the cannula. In one aspect, the finger tabs 21 are integral with the arms, snaps or hooks extending laterally from the outer periphery of the seal cover 9 and perpendicular to the longitudinal axis of the seal cover 9. The finger tabs 21 provide a generally flat surface area to be manipulated or grasped to attach and/or detach the seal assembly 5 to the cannula 6. In one aspect, slot tabs 41 extend from the seal cover and are coupled to the hooks and/or arms 22 to provide an access or mating portion for detaching the seal assembly by utilizing a flat instrument or grasper. For example, a slot tab is provided having a projection extending therefrom to engage and secure the seal cover 9 to the cannula 6. A cavity defined by walls extending laterally from the seal cover receives a flat or similarly configured mating instrument corresponding to the cavity configuration to cause the projection to move outward resulting in the disengagement of the projection from the cannula ledge or ledges.

The connection of the seal assembly 5 to the cannula 6 can also serve to prevent or resist removal of the seal assembly relative to the cannula 6 when the seal assembly is rotated. For example, the projections engaging the mating flange of the cannula 6 prevent or resist the seal assembly 5 from being twisted off of the cannula 6 during manipulation of the seal assembly, e.g., when the seal assembly is rotated. The seal assembly and cannula attachment can also be threaded and/or have bayonet lock connections to attach the seal assembly 5 to the cannula 6. However, the attachment of the projections 22 of cover 9 with the cannula 6 prevents unintended detachment of the seal assembly from the cannula that may result by inadvertently rotating the seal assembly relative to the cannula during manipulation of inserted instrumentation or manipulation of the trocar. An unintended detachment of a seal assembly 5 from a cannula 6 during a surgical procedure may result in a loss of insufflation gas, a loss of visibility of the operative area, a delay in the procedure, and/or other potential surgical issues.

One or more hooks or projections 28 extend from the sleeve 11 to secure the septum seal 8 and/or zero seal 7 to the seal cover. In one aspect, the septum seal 8 and/or zero seal 7 has a lip or flange for mating with the hook 28 extending from the seal cover 9. In one aspect, disposed along an inner periphery of the seal cover 9 are one or more slots 27. In one aspect, the septum seal 8 and/or zero seal 7 has a lip or flange for mating with or extending into the one or more slots 27. These connections assist in coupling and sealing the seal cover/sleeve to the septum seal 8 and/or zero seal 7 and the seal assembly 5 to the cannula 6. These connections also assist in maintaining the axial position of the seal assembly on the cannula and prevent unintended dislodgment of the seal assembly from the cannula. These connects are also further fortified by compression as the seal assembly is mated with the cannula.

The projection 28 from the seal sleeve 11 in conjunction with the seal cover 9 in one aspect are resilient and flex during attachment of the seal cover/sleeve to the septum seal 8, zero seal 7 and/or the cannula 6. In one aspect, the sleeve and projection bias portions of the septum seal 8 and/or zero seal 7 along an opposing wall of the seal cover to enhance attachment of the septum seal and/or zero seal to the seal cover 9 or seal sleeve 11. The wall and projection in one aspect also biases portions of the septum seal 8 and/or zero seal 7 towards the cannula 6 when the seal assembly 5 is connected to the cannula 6. In one aspect, the slots 27 along the surface of the seal cover 9 provide access to the septum seal 8 and/or zero seal 7 to assist in assembly and/or removal of the septum seal/zero seal from the cannula 6.

The seal assembly in one aspect comprises three components. Using just three components reduces manufacturing costs and assembly time and eases use or operation of the seal assembly and cannula. In one aspect, the seal assembly comprises a polyethylene seal cover and sleeve, a compression molded polyisoprene septum seal, and a compression molded polyisoprene duckbill valve. To assemble the seal assembly, the septum seal is nested into the duckbill valve. The cover is fitted onto the septum seal and duckbill valve. The seal assembly is snap fitted onto the cannula. In one aspect, the seal cover/sleeve comprises polyethylene in which the sleeve extends from the seal cover and is nested into the septum seal. The sleeve prevents instruments from catching and/or tearing the septum seal. The sleeve can also prevent the septum seal from inverting during instrument withdrawal. The sleeve can also reduce the instrumentation drag force. In one aspect, the zero seal is a double duckbill valve and/or a single duckbill.

In one aspect, the zero seal 7 is generally cylindrical having a proximal portion coupled to the proximal portion of the septum seal. The distal portion of the zero seal has one or more leaflets extending from the cylindrical body portion radially converge towards the center or longitudinal axis of the zero seal and seal assembly. The leaflets seal the passage or channel of the cannula and seal assembly when no instrument is inserted. In one aspect, the septum seal 8 is generally cylindrical having a proximal portion coupled to the proximal portion of the zero seal. The distal portion of the zero seal is generally conical converging to an aperture at the center or longitudinal axis of the seal assembly. The cylindrical body portion of the septum seal 8 is in contact with the cylindrical body portion of the zero seal. In one aspect, the conical portion of the septum seal 8 is not in contact with the one or more leaflets of the zero seal. The separation between the distal portions of the septum and zero seals assists in avoiding binding of the seals or interference between the seals as an instrument is inserted and/or withdrawn. As such, in one aspect, the length of the zero seal 7 is generally longer than the septum seal 8.

The cylindrical portion of the sleeve 11 is in contact with the cylindrical body portion of the septum seal. The distal leaflets 12 of the sleeve are operationally in contact with the conical portion of the septum seal 8. In one aspect, the leaflets overlap each other preventing gaps between the leaflets as an instrument is inserted and the leaflets pivot or flex thereby continually protecting the seal. Also, the leaflets overlapping reduce potential binding of the leaflets and eases manufacturing of the sleeve and assembly of the seal assembly. The leaflets distal portions in one aspect contact the aperture of the septum seal. The leaflets extending from the sleeve act as living hinges capable of flexing and/or pivoting radially outward from the center of the sleeve. As such, the leaflets are flexible and yet rigid to protect the septum seal. The sleeve 11 is also similarly rigid thereby protecting the septum seal.

The outer diameter of the septum seal 8 generally corresponds to the inner diameter of the zero seal 7. The inner diameter of the septum seal generally corresponds to the outer diameter of the sleeve 11 and is generally smaller than the inner diameter of the zero seal. In one aspect, the septum seal is fixed to the zero seal that is fixed to the seal cover.

Accordingly, the present invention provides a surgical access device with a removable seal assembly. Although this invention has been described in certain specific embodiments, many additional modifications and variations would be apparent to those skilled in the art. It is therefore to be understood that this invention may be practiced otherwise than specifically described, including various changes in the size, shape and materials, without departing from the scope and spirit of the present invention. Thus, embodiments of the present invention should be considered in all respects as illustrative and not restrictive, the scope of the present invention to be determined by the appended claims and their equivalents rather than the foregoing description.

1. A trocar seal comprising:
an instrument seal having a proximal end and a distal end;
a zero seal having a proximal end and a distal end, the proximal end of the zero seal being connected to the proximal end of the instrument seal;
a cover connected to the proximal end of the instrument seal; and
a sleeve monolithically formed with the cover and extending from the cover towards the distal end of the instrument seal.

2. The trocar seal of claim 1 wherein the zero seal is generally cylindrical and the instrument seal is generally cylindrical with the distal end of the instrument seal being generally conical and an aperture extends through the distal end of the instrument seal.

3. The trocar seal of claim 2 wherein the instrument seal has a length corresponding to a length of the zero seal and the instrument seal is coaxially aligned with the zero seal.

4. The trocar seal of claim 1 wherein the sleeve has a body portion extending in a longitudinal direction relative to the cover with the cover having a projection extending in a direction traverse to the longitudinal direction of the body portion of the sleeve.

5. The trocar seal of claim 1 further comprising a cannula with a proximal end and a distal end and a flange extending from the proximal end of the cannula, the cover peripherally covering the proximal end of the cannula and having a projection connectable to the flange on the cannula.

6. The trocar seal of claim 1 wherein the cover has peripherally spaced snaps extending from the cover.

7. The trocar seal of claim 6 wherein the snaps are resilient and arranged to pivot and flex away from the longitudinal axis of the cover.

8. The trocar seal of claim 1 wherein the sleeve is less compliant than the instrument seal.

9. A trocar seal comprising:
an instrument seal having a proximal end and a distal end;
a zero seal having a proximal end and a distal end, the proximal end of the zero seal being connected to the proximal end of the instrument seal;
a cover connected to the proximal end of the instrument seal, the cover having a substantially cylindrical sleeve extending from the cover and into the instrument seal and the cover having snaps extending parallel to the longitudinal axis of the cover and having projections extending orthogonal from the snaps towards the instrument seal.

10. The trocar seal of claim 9 wherein the cover further comprises tabs extending orthogonally from the snaps away from the instrument seal.

11. The trocar seal of claim 9 wherein the snaps are resilient and are arranged to pivot away from the cover and the instrument seal.

12. The trocar seal of claim 9 wherein the sleeve has projections extending radially from the sleeve and connect with the instrument seal.

13. The trocar seal of claim 9 further comprising a cannula and wherein the cover is removably and directly connected to the cannula.

14. The trocar seal of claim 9 wherein the cannula has a lumen extending from the proximal end to the distal end and a enlarged cavity on the proximal end of the cannula with a diameter larger than a diameter of the lumen of the cannula, the proximal end of the instrument seal having a diameter smaller than the diameter of the proximal end of the enlarged cavity and the proximal end of the zero seal having a diameter smaller than the diameter of the proximal end of the enlarged cavity.

15. The trocar seal of claim 9 wherein the cover has at least one slot in the periphery of the cover.

16. The trocar seal of claim 9 wherein the projections restrict axial movement of the cover and allow rotational movement of the cover.

17. A trocar seal comprising:
an instrument seal;
a zero seal nested in the instrument seal;
a cover connected to the instrument seal having means for protecting the instrument seal, the cover arranged to be removably connected to a cannula by a means for restricting axial movement of the cover and for allowing rotational movement of the cover relative to the cannula.

18. The trocar seal of claim 17 wherein the means for restricting axial movement of the cover and allowing rotational movement of the cover comprises at least one resilient arm extending from the cover.

19. The trocar seal of claim 17 wherein the means for allowing rotational movement allows the cover to rotate 360 degrees.

20. The trocar seal of claim 17 wherein the cover further comprises at least one tab extending from the cover in a direction traverse to the longitudinal axis of the instrument seal and at least one gusset extending from the cover to the at least one tab.
